(19) United States
(12) Patent Application Publication
(10) Pub. No.: US 2016/0113526 A1
(43) Pub. Date: Apr. 28, 2016

(54) GENERATING USER INFORMATION FROM AUTONOMIC NERVOUS SYSTEM PARAMETERS

(71) Applicant: DyAnsys, Inc., San Mateo, CA (US)

(72) Inventors: Srini Nageshwar, Los Gatos, CA (US); Melvyn J. Lafitte, Prevessin-Moens (FR)

(21) Appl. No.: 14/922,049

(22) Filed: Oct. 23, 2015

Related U.S. Application Data
(60) Provisional application No. 62/067,955, filed on Oct. 23, 2014.

(51) Int. Cl. A61B 5/026; A61B 5/024; A61B 5/00; A61B 5/0295

(52) U.S. Cl. CPC A61B 5/0261; A61B 5/0295; A61B 5/02433; A61B 5/4041; A61B 5/7278; A61B 5/721; A61B 5/4884; A61B 5/7455

(57) ABSTRACT

Techniques are disclosed for generating user information from autonomic nervous system (ANS) parameters and continuous ANS monitoring. Some embodiments may provide a wearable device, comprising: a photoplethysmography (PPG) sensor; a motion sensor; and processing circuitry configured to: determine one or more monitoring conditions; determine whether the one or more monitoring conditions are satisfied based on monitoring motion data from the motion sensor; capture the blood flow data with the PPG sensor when the one or more monitoring conditions are satisfied; determine time-interval data indicating times between pulses based on the blood flow data; determine an ANS condition based on the time-interval data. The blood flow data and/or time-interval data may be captured and aggregated from multiple discontinuous periods of time when monitoring conditions are satisfied. The processing circuitry may be further configured to control output devices for presentation of the ANS condition(s), such as an actuator of the wearable device.

### CROSS-REFERENCE TO RELATED APPLICATIONS

The present application claims the benefit of U.S. Provisional Patent Application No. 62/067,955, titled “Generating User Information from Autonomic Nervous System Parameters,” filed Oct. 23, 2014, which is incorporated herein by reference in its entirety.

The present application is related to U.S. Pat. No. 7,092,849, which is incorporated herein by reference in its entirety.

### TECHNICAL FIELD

The example embodiments of the present invention generally relate to techniques for autonomic nervous system (ANS) monitoring.

### BACKGROUND

Evaluating the ANS health of a user conventionally requires that the user remain substantially stationary, or in a supine or quasi-supine position. This is because more strenuous physical activity causes heart rate to increase, which can distort heartbeat measurements used in evaluating ANS conditions or health, and because ANS analysis may be unreliable without a sufficient large data set. As such, conventional techniques require users to set aside blocks of inactive down time for ANS monitoring. Such a requirement inconveniences users, resulting in lower of participation, and degraded user health over time. In this and other regards discussed herein, areas for improving current techniques have been identified.

### BRIEF SUMMARY

Through applied effort, ingenuity, and innovation, solutions to improve ANS monitoring have been realized and are described in connection with embodiments of the present invention. For example, some embodiments may provide for a wearable device, comprising: a photoplethysmography (PPG) sensor configured to generate blood flow data of user; a motion sensor configured to generate motion data indicating motion of the user; and processing circuitry configured to: determine one or more monitoring conditions; determine whether the one or more monitoring conditions are satisfied based on monitoring the motion data; capture the blood flow data when the one or more monitoring conditions are satisfied; determine time-interval data indicating times between pulses s based on the blood flow data; and determine an autonomic nervous system condition based on the time-interval data.

In some embodiments, the processing circuitry configured to capture the blood flow data when the one or more monitoring conditions are satisfied may include the processing circuitry being configured to: capture first blood flow data within a first time interval when the one or more monitoring conditions are satisfied; capture second blood flow data within a second time interval when the one or more monitoring conditions are satisfied, wherein the first time interval and the second time interval are discontinuous; and determine the blood flow data based on aggregating the first blood flow data and the second blood flow data.

In some embodiments, the processing circuitry configured to capture the blood flow data when the one or more monitoring conditions are satisfied may include the processing circuitry being configured to capture blood flow data within discontinuous time intervals until a predetermined amount of the blood flow data sufficient for the autonomous nervous condition determination has been captured while the one or more monitoring conditions are satisfied.

In some embodiments, the one or more monitoring conditions include at least one of: the user being in a quasi-supine position; the user performing spontaneous respiration; or a lack of user motion.

In some embodiments, the processing circuitry may be further configured to: determine sympathetic component values based on the time-interval data; determine sympathetic index values based on the sympathetic component values; determine parasympathetic component values based on the time-interval data; and determine a parasympathetic index values based on the time-interval data.

In some embodiments, the processing circuitry configured to determine the autonomic nervous system condition may include the processing circuitry being configured to determine an autonomic dysfunction indicating a lack of coupling between the sympathetic index values and the parasympathetic index values.

In some embodiments, the processing circuitry configured to determine the autonomic nervous system condition may include the processing circuitry being configured to determine a wellness value indicating a desirable coupling between the sympathetic index values and the parasympathetic index values.

In some embodiments, the one or more monitoring conditions include the user being stationary. The wearable device may further include an actuator configured to provide a haptic output. The processing circuitry configured to determine the autonomic nervous system condition may include the processing circuitry being configured to: determine a stress value based on the sympathetic index values; determine a stress value threshold; and control the actuator to provide the haptic output when the stress value satisfies the stress value threshold.

In some embodiments, the wearable device may further include an actuator configured to provide a haptic output. The processing circuitry may be further configured to control the actuator to provide the haptic output based on the autonomic nervous system condition.

In some embodiments, the photoplethysmography (PPG) sensor may include: one or more light emitting diodes (LEDs) configured to provide light on the user's skin; and one or more photodiodes configured to measure changes in absorption or reflection of the light by the user's skin to generate the blood flow data of the user.

In some embodiments, the one or more LEDs may include at least one of: a green LED; a red LED; a near-infrared LED; an infrared LED; or a blue LED.

In some embodiments, the one or more LEDs may include a green LED and an infrared LED. The processing circuitry may be further configured to: capture the blood flow data when the one or more monitoring conditions are satisfied using the green LED to provide the light on the user's skin; and capture second blood flow data when the one or more monitoring conditions fails to be satisfied using the infrared LED to provide the light on the user's skin.

Some embodiments may provide for a method for autonomic nervous system (ANS) monitoring. The method may include: determining, by processing circuitry of an apparatus, one or more monitoring conditions; determining, by the processing circuitry, whether the one or more monitoring conditions are satisfied based on monitoring motion data captured by a motion sensor of a wearable device; controlling, by the processing circuitry, a photoplethysmography (PPG) sensor of the wearable device to capture blood flow data when the one or more monitoring conditions are satisfied; determining, by the processing circuitry, time-interval data indicating times between pulses based on the blood flow data; and determining, by the processing circuitry, an autonomic nervous system condition based on the time-interval data.

In some embodiments, controlling the PPG sensor to capture the blood flow data when the one or more monitoring conditions are satisfied may include: controlling the PPG sensor to capture first blood flow data within a first time interval when the one or more monitoring conditions are satisfied; controlling the PPG sensor to capture second blood flow data within a second time interval when the one or more monitoring conditions are satisfied, wherein the first time interval and the second time interval are discontinuous; and determining the blood flow data based on aggregating the first blood flow data and the second blood flow data.

In some embodiments, controlling the PPG sensor to capture the blood flow data when the one or more monitoring conditions are satisfied may include controlling the PPG sensor to capture blood flow data within discontinuous time intervals until a predetermined amount of the blood flow data sufficient for the autonomous nervous condition determination has been captured while the one or more monitoring conditions are satisfied.

In some embodiments, the one or more monitoring conditions include at least one of: the user being in a quasi-supine position; the user performing spontaneous respiration; or a lack of user motion.

In some embodiments, the method may further include, by the processing circuitry: determining sympathetic component values based on the time-interval data; determining sympathetic index values based on the sympathetic component values; determining parasympathetic component values based on the time-interval data; and determining a parasympathetic index values based on the time-interval data.

In some embodiments, determining the autonomic nervous system condition may include determining an autonomic dysfunction indicating a lack of coupling between the sympathetic index values and the parasympathetic index values.

In some embodiments, determining the autonomic nervous system condition may include determining a wellness value indicating a desirable coupling between the sympathetic index values and the parasympathetic index values.

In some embodiments, the one or more monitoring conditions include the user being stationary. The wearable device further may further include an actuator configured to provide a haptic output. Determining the autonomic nervous system condition may include: determining a stress value based on the sympathetic index values; determining a stress value threshold; and controlling the actuator to provide the haptic output when the stress value satisfies the stress value threshold.

In some embodiments, the wearable device further includes an actuator configured to provide a haptic output. The method further includes, by the processing circuitry, controlling the actuator to provide the haptic output based on the autonomic nervous system condition.

In some embodiments, the photoplethysmography (PPG) sensor may include: one or more light emitting diodes (LEDs) configured to provide light on the user's skin; and one or more photodiodes configured to measure changes in absorption or reflection of the light by the user's skin to generate the blood flow data of the user.

In some embodiments, the one or more LEDs may include at least one of: a green LED; a red LED; a near-infrared LED; an infrared LED; or a blue LED.

In some embodiments, the one or more LEDs include a green LED and an infrared LED. The processing circuitry may be further configured to: capture the blood flow data when the one or more monitoring conditions are satisfied using the green LED to provide the light on the user's skin; and capture second blood flow data when the one or more monitoring conditions fails to be satisfied using the infrared LED to provide the light on the user's skin.

In some embodiments, the apparatus including the processing circuitry may be one of: the wearable device; a user device tethered to the wearable device; or a server connected to the wearable device via the Internet.

Some embodiments may include one or more machines, such as an apparatus and/or system, configured to implement the methods and/or other functionality discussed herein. For example, the machine may include one or more processors and/or other machine components configured to implement the functionality discussed herein based on instructions and/or other data stored in memory and/or other non-transitory computer readable media

These characteristics as well as additional features, functions, and details are described below. Similarly, corresponding and additional embodiments are also described below.

### BRIEF DESCRIPTION OF THE DRAWING(S)

Having thus described the example embodiments of the present invention in general terms, reference will now be made to the accompanying drawings, which are not necessarily drawn to scale, and wherein:

FIG. 1 shows an example of a system in accordance with some embodiments;

FIG. 2 shows an example of a system in accordance with some embodiments;

FIG. 3 shows a schematic block diagram of an example of a wearable device in accordance with some embodiments;

FIG. 4 shows a schematic block diagram of example circuitry in accordance with some embodiments; and

FIG. 5 shows an example of a method of monitoring the autonomic nervous system (ANS) of a user in accordance with some embodiments.

### DETAILED DESCRIPTION

Various embodiments will now be described more fully with reference to the accompanying drawings, in which some, but not all embodiments of the disclosure are shown. This disclosure may be embodied in many different forms and should not be construed as limited to the embodiments set forth; rather, these example embodiments are provided so that this disclosure will be thorough and complete, and will fully convey the scope of the disclosure to those skilled in the art. Like numbers refer to like elements throughout.

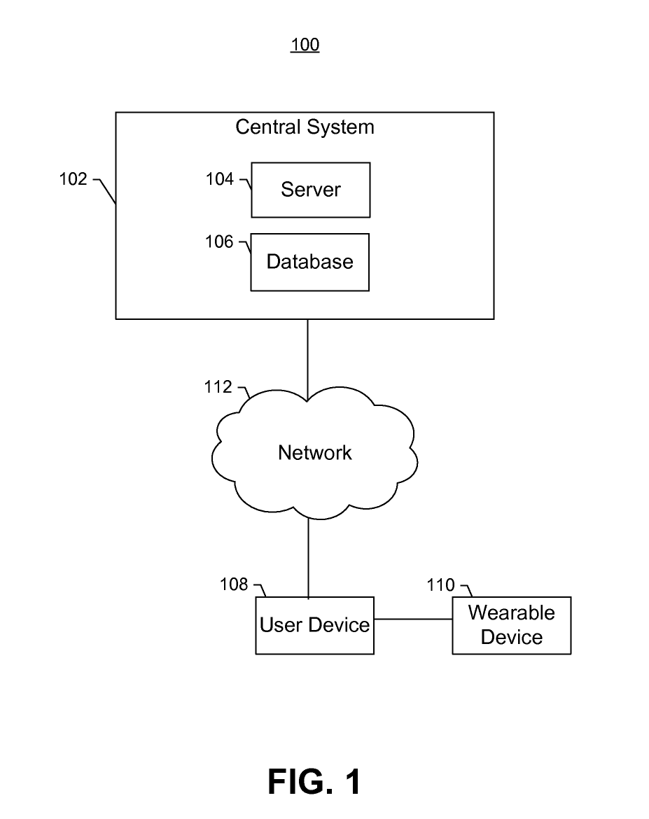

FIG. 1 shows an example computing system 100, in accordance with some embodiments. A user may access a health monitoring service provided by a central system 102 via a network 112 (e.g., the Internet) using a user device 108 and/or a wearable device 110. Moreover, the central system 102 may comprise a server 104 and a database 106. Although a single user device 108 and wearable device 110 is shown in FIG. 1, the system 100 may include multiple user devices 108 and wearable devices 110 each associated with a user.

The user device 108 may be any computing device as known in the art and operated by a user. The user device 108 may include a mobile device, such as a smartphone, netbook, tablet computer, laptop computer, or the like.

The wearable device 110 may be a mobile device that is configured to be worn on, attached to, carried on, or implanted within the user, such as an electronic smartwatch, wrist band, glasses, implantable device, etc. In some embodiments, the wearable device 110 may be wirelessly tethered to the user device 108, such as via a personal area network (PAN). The PAN may be configured to use a Bluetooth protocol for data transmissions between the user device 108 and the wearable device 110. However, other types of connections or protocols may be used, such as a wired connection and/or other wireless technologies such as Infrared Data Association (IrDA), near field communication (NFC), wireless USB, Z-wave, ZigBee, etc.

When tethering is used, the wearable device 110 may include effectively operate as a light client with input and output circuitry/devices for the host user device 108. Some example input devices of the wearable device 110 may include a touch screen, button(s), motion sensor, and photoplethysmography (PPG) sensor. Some example output devices of the wearable device 110 may include a display, speaker, and actuator. The wearable device 110 may also include a data bus or communication fabric that provides for data transmissions between the wearable device 110 and the user device 108. Here, the user device 108 may include processing circuitry configured to receive data inputs from the wearable device 110, process the received data as discussed herein to facilitate user health monitoring, and provide outputs to the wearable device 110 for communication with the user.

In embodiments where the user device 108 and/or wearable device 110 may execute “apps” to perform the functionalities discussed herein, and interact with each other or the central system 102. Such apps are typically designed to execute on mobile devices. For example, an app may be provided that executes on mobile device operating systems such as Apple Inc.'s iOS®, Google Inc.'s Android®, or Microsoft Inc.'s Windows 8®. These platforms typically provide frameworks that allow apps to communicate with one another and with particular hardware and software components of mobile devices, such as via application programming interfaces (APIs) provided by the mobile device operating system.

The server 104 may be embodied as a single computer or multiple computers. The server 104 may provide for aggregation of user data from multiple user devices 108 and/or wearable devices 110. The aggregation of user data may provide for relative health position of a user to other users in a population, machine learning for correlations between measured data and health outcomes (e.g., autonomic nervous system conditions), and correlations between health outcomes and treatments. In some embodiments, the server 104 may be further configured to operate has a host device for a thin user devices 108, and thus may include processing circuitry configured to analyze measured data, such as blood flow data captured by the PPG sensor of the wearable device 110, to determine autonomic nervous system conditions. The server 104 may be further configured to control outputs of the wearable device 110 for user communication.

Database 106 may be embodied as a data storage device such as a Network Attached Storage (NAS) device or devices, or as a separate database server or servers. The database 106 includes information accessed and stored by the server 104 to facilitate the operations of the central system 102. For example, the database 106 may include, without limitation, user blood flow data, associated user ANS conditions, user account information (e.g., profiles, login data, etc.), etc.

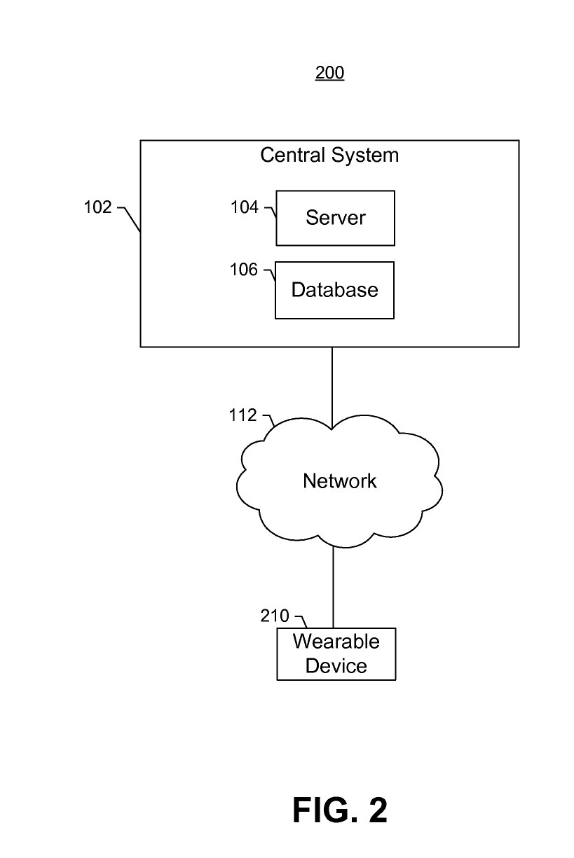

FIG. 2 shows an example computing system 200, in accordance with some embodiments. The discussion above with respect to computing system 100 may be applicable to computing system 200. However, the user device 108 is omitted and there is no tethering with the wearable device 210. Instead, the wearable device 210 may be a standalone device that includes the processing circuitry configured to process blood flow data. Furthermore, the wearable device 210 may communicate with the central system 102 via the network 112 without the intervening user device 108. In some embodiments of the computing system 200, the wearable device 210 may be a thin client that communicates the server 104 configured to process the measured data inputs and control outputs of the wearable device 210.

Advantageously, performing the data processing on the user device 108 may allow for a smaller, lighter, cheaper, and more power efficient wearable device 110 (e.g., resulting in longer battery life between recharges). However, performing the data processing on the wearable device 210 allows for standalone operation, and the user does not need to carry both a user device and the wearable device 210 to receive continuous monitoring.

In some embodiments, the role of the central system 102 may be further reduced to provide functionality without requiring a central system. Here, the user device 108 and/or wearable device 110/210 may be configured to perform the functionality discussed herein, with the central system providing the applications that can be executed by the user device 108 and/or wearable device 110/210 to configure these devices to perform the functionality discussed herein.

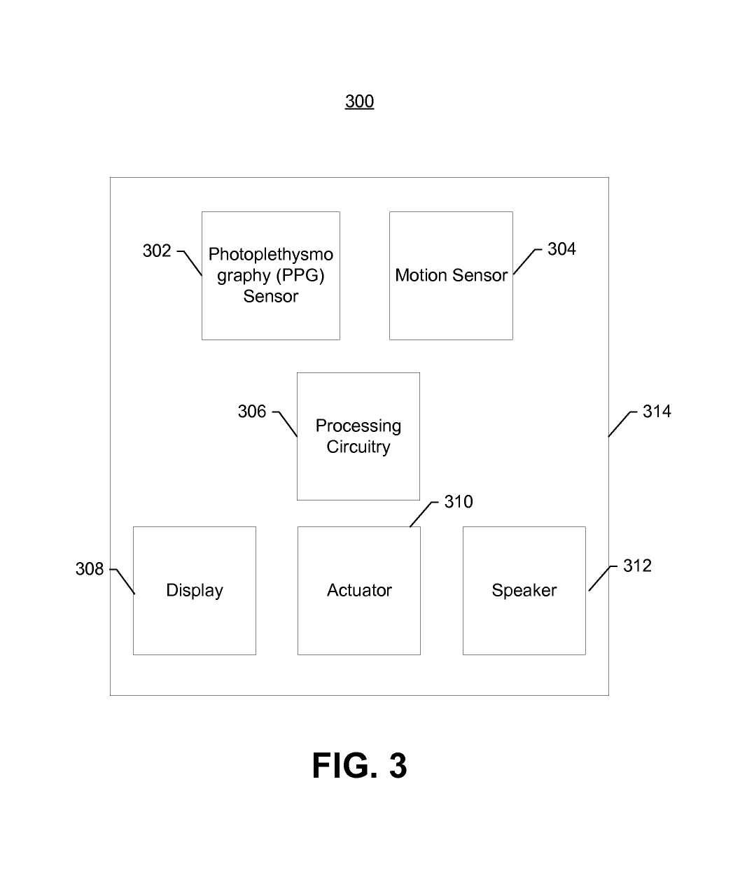

FIG. 3 shows a schematic block diagram of an example of a wearable device 300, in accordance with some embodiments. The wearable device 300 may include a photoplethysmography (PPG) sensor 302, a motion sensor 304, processing circuitry 306, display 308, actuator 310, speaker 312, and a housing 314.

The PPG sensor 302 operates based on the physical principle that blood (e.g., being red) reflects and absorbs light differently from surrounding tissue. For example, blood absorbs more red light and reflects green light more than the surrounding tissue, and thus changes in the level of light absorption over time indicates changes in the amount of blood flow over time, and thus the heartbeat rate. The PPG sensor 302 may include one or more light emitting diodes (LEDs) configured to provide light on the user's skin when the wearable device 300 is worn on the user, and one or more photodiodes configured to measure changes in absorption or reflection of the light by the user's skin to generate blood flow data. For example, when the wearable device is an electronic watch, the PPG sensor 302 may be located on the back side of the housing 314 to interface with the skin, and on the opposite of the housing 314 relative to the display 308.

The one or more LEDs may include a green LED (e.g., around 530 nm wavelength), a red LED (e.g., around 660 nm wavelength), a near-infrared LED, an infrared LED (e.g., around 940 nm wavelength), or a blue LED (around 470 nm wavelength). However, the PPG sensor 302 may include one or more LEDs of various wavelengths. The type of LED(s) used may be optimized based on performance and/or power consumption, and may further depend on the location where the wearable device is designed to be worn on the user. For example, studies indicate that the green LED may provide for the optimal accuracy for heartbeat measurement for wearable devices wrist (e.g., by providing for better absorption by blood, and resulting in better signal-to-noise ratios), and the red or infrared LED may provide for the optimal accuracy for devices worn on the finger(s). The green LED, having a higher frequency of emitted light, may also consumer more power than the red or infrared LED.

In some embodiments, multiple different LEDs may be used at different times to optimize device performance and power consumption. For example, the PPG sensor 302 may include a green LED and a red/infrared LED. The green LED may be activated when accuracy and precision are prioritized, such as when one or more monitoring conditions (e.g., the user is in a quasi-supine position, performing spontaneous respiration, and/or is not in motion) for the ANS are satisfied, and the resulting blood flow data may be captured and stored. Conversely, when the one or more monitoring conditions are not satisfied, the lower power consumption red LED may be activated and the green LED may be deactivated to provide continuous and/or more intermittent monitoring (which may or may not be captured or recorded to a data storage).

The one or more photodiodes of the PPG sensor 302 may be sensors configured to measure changes in absorption or reflection of the light by the user's skin to generate the blood flow data. For example, a first portion of the light generated by the one or more LEDs may be absorbed by the skin, vasculature, and/or blood, and a second portion of the light may be reflected back to the one or more photodiodes which measure light intensity. The blood flow data may represent a signal that corresponds with the measured light intensity. For example, when green LED light is used, higher blood flow may correspond with greater measured light intensities because more green light is reflected back to the photodiode. In some embodiments, where multiple LEDs are used, multiple photodiodes may be used that each measure light intensities from a respective LED. In some embodiments, the PPG sensor may include a magneto-plethysmographic sensor in alternative or addition to the photo-plesthysmographic sensor.

The motion sensor 304 of the wearable device 300 may be configured to generate motion data indicating motion of the user when the wearable device 300 is worn by the user. For example, the motions sensor 304 may include a 3D digital gyroscope and accelerometer. In some embodiments, the motion sensor 304 may be a single component a six axis sensor that measures roll, pitch and yaw (gyroscope). In some embodiments, multiple motion sensors may be used, such as a higher power consumption but higher sensitivity six axis accelerometer and a lower power consumption but lower power consumption three axis accelerometer. In some embodiments, the motion sensor may include a multi-axis sensor (typically, 6-axis accelerometer), a digital gyroscope and a magnetometer. The wearable device 300 may further include other input devices, such as a touch screen, microphone, camera, fingerprint reader, etc.

The display 308, actuator 310, and speaker 312 may be output devices of the wearable device 300. The display 308 may include a flexible organic LED (OLED) display covered by a sapphire crystal or ion-x cover glass. However, other types of displays may be used such as liquid crystal display (LCD), LED, etc.

The actuator 310 may be configured to provide a haptic output as user feedback. The actuator 310 may be a linear actuator that creates mechanical motion along a single axis in response to an electrical control signal, thereby causing a vibration or motion of the wearable device 310 that can be detected by the user. In some embodiments, a rotating-mass vibration motor actuator may be used in alternative or addition to the linear actuator.

The processing circuitry 306 may be configured to provide the functionality discussed herein with respect to control of the various input and output devices, and interfacing with the user device 108 or central system 102. In some embodiments, such as when the wearable device 300 is a standalone device and/or not tethered to the user device 108, the processing circuitry 306 may be further configured to perform the processing functionality discussed herein with respect to health and ANS monitoring based on received data inputs.

The components of the wearable device 300 may be attached with or located within the housing 314. The housing 314 may further include or be attached with a mechanical user interfacing element, such as a strap or wristband when the wearable device is a smartwatch.

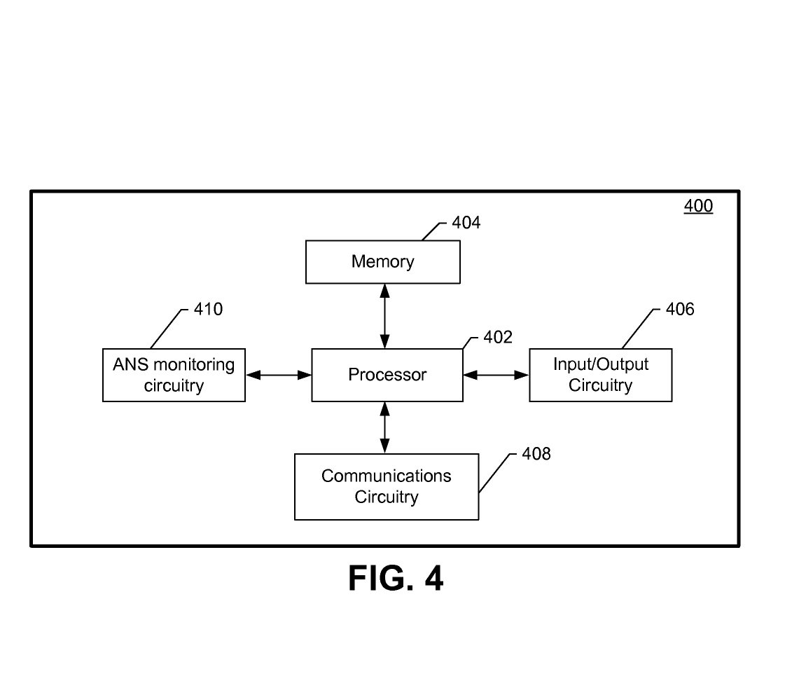

The wearable device 110/210/300, user device 108, server 104, or database 106 may be embodied by one or more computing systems or devices, such as apparatus 400 shown in FIG. 4. As illustrated in FIG. 4, the apparatus 400 may include a processor 402, a memory 404, an input/output circuitry 406, a communications circuitry 408, and an autonomic nervous system (ANS) monitoring circuitry 410. The apparatus 400 may be configured to execute the operations described herein. Although these components 402-410 are described with respect to functional limitations, it should be understood that the particular implementations necessarily include the use of particular, non-generic hardware. It should also be understood that certain of these components 402-410 may include similar or common hardware. For example, two sets of circuitry may both leverage use of the same processor, network interface, storage medium, or the like to perform their associated functions, such that duplicate hardware is not required for each set of circuitry. The use of the term “circuitry” as used herein with respect to components of the apparatus should therefore be understood to include particular hardware configured to perform the functions associated with the particular circuitry as described herein.

The term “circuitry” should be understood broadly to include hardware and, in some embodiments, software for configuring the hardware. For example, in some embodiments, “circuitry” may include processing circuitry, storage media, network interfaces, input/output devices, and the like. In some embodiments, other elements of the apparatus 400 may provide or supplement the functionality of particular circuitry. For example, the processor 402 may provide processing functionality, the memory 404 may provide storage functionality, the communications circuitry 408 may provide network interface functionality, and the like.

In some embodiments, the processor 402 (and/or co-processor or any other processing circuitry assisting or otherwise associated with the processor) may be in communication with the memory 404 via a bus for passing information among components of the apparatus 400. The memory 404 may be non-transitory and may include, for example, one or more volatile and/or non-volatile memories. In other words, for example, the memory may be an electronic storage device (e.g., a computer readable storage medium). The memory 404 may be configured to store information, data, content, applications, instructions, or the like, for enabling the apparatus to carry out various functions in accordance with example embodiments of the present invention.

The processor 402 may be embodied in a number of different ways and may, for example, include one or more processing devices configured to perform independently. Additionally or alternatively, the processor may include one or more processors configured in tandem via a bus to enable independent execution of instructions, pipelining, and/or multithreading. The use of the term “processing circuitry” may be understood to include a single core processor, a multi-core processor, multiple processors internal to the apparatus, and/or remote or “cloud” processors.

In an example embodiment, the processor 402 may be configured to execute instructions stored in the memory 404 or otherwise accessible to the processor. Alternatively or additionally, the processor 402 may be configured to execute hard-coded functionality. As such, whether configured by hardware or software methods, or by a combination thereof, the processor may represent an entity (e.g., physically embodied in circuitry) capable of performing operations according to various embodiments while configured accordingly. Alternatively, as another example, when the processor 402 is embodied as an executor of software instructions, the instructions may specifically configure the processor to perform the algorithms and/or operations described herein when the instructions are executed.

In some embodiments, the apparatus 400 may include input/output circuitry 406 that may, in turn, be in communication with processor 402 to provide output to the user and, in some embodiments, to receive an indication of a user input and/or measured data input. The input/output circuitry 406 may comprise a user interface and may include a display and may comprise a mobile application, a web user interface, a client device, or the like. The input/output circuitry 406 may include input devices such as the PPG sensor, motion sensor, touch screen, microphone, camera, dial (or “digital crown”), button, etc. and output devices such as the display, actuator, speaker, etc. In some embodiments, the input/output circuitry 406 may also include other input/output devices such as a keyboard, a mouse, a joystick, soft keys, etc. The processor and/or user interface circuitry comprising the processor may be configured to control one or more functions of one or more user interface elements through computer program instructions (e.g., software and/or firmware) stored on a memory accessible to the processor (e.g., memory 404, and/or the like).

The communications circuitry 408 may be any means such as a device or circuitry embodied in either hardware or a combination of hardware and software that is configured to receive and/or transmit data from/to a network and/or any other device, circuitry, or module in communication with the apparatus 400. In this regard, the communications circuitry 408 may include, for example, a network interface for enabling communications with a wired or wireless communication network. For example, the communications circuitry 408 may include one or more network interface cards, antennae, buses, switches, routers, modems, and supporting hardware and/or software, or any other device suitable for enabling communications via a network. Additionally or alternatively, the communication interface may include the circuitry for interacting with the antenna(s) to cause transmission of signals via the antenna(s) or to handle receipt of signals received via the antenna(s).

Circuitry 400 may further include the ANS monitoring circuitry 410 configured to provide the functionality discussed herein with respect to monitoring health and ANS conditions based on blood flow data from the PPG sensor. Circuitry 410 may utilize processing circuitry, such as the processor 402, to perform these actions. However, it should also be appreciated that, in some embodiments, circuitry 410 may include a separate processor, specially configured field programmable gate array (FPGA), or application specific interface circuit (ASIC). Circuitry 410 may therefore be implemented using hardware components of the apparatus configured by either hardware or software for implementing these planned functions.

As will be appreciated, any such computer program instructions and/or other type of code may be loaded onto a computer, processor or other programmable apparatus's circuitry to produce a machine, such that the computer, processor other programmable circuitry that execute the code on the machine create the means for implementing various functions, including those described herein.

As described above and as will be appreciated based on this disclosure, various embodiments may be configured as methods, mobile devices, backend network devices, and the like. Accordingly, embodiments may comprise various means including entirely of hardware or any combination of software and hardware. Furthermore, embodiments may take the form of a computer program product on at least one non-transitory computer-readable storage medium having computer-readable program instructions (e.g., computer software) embodied in the storage medium. Any suitable computer-readable storage medium may be utilized including non-transitory hard disks, CD-ROMs, flash memory, optical storage devices, or magnetic storage devices.

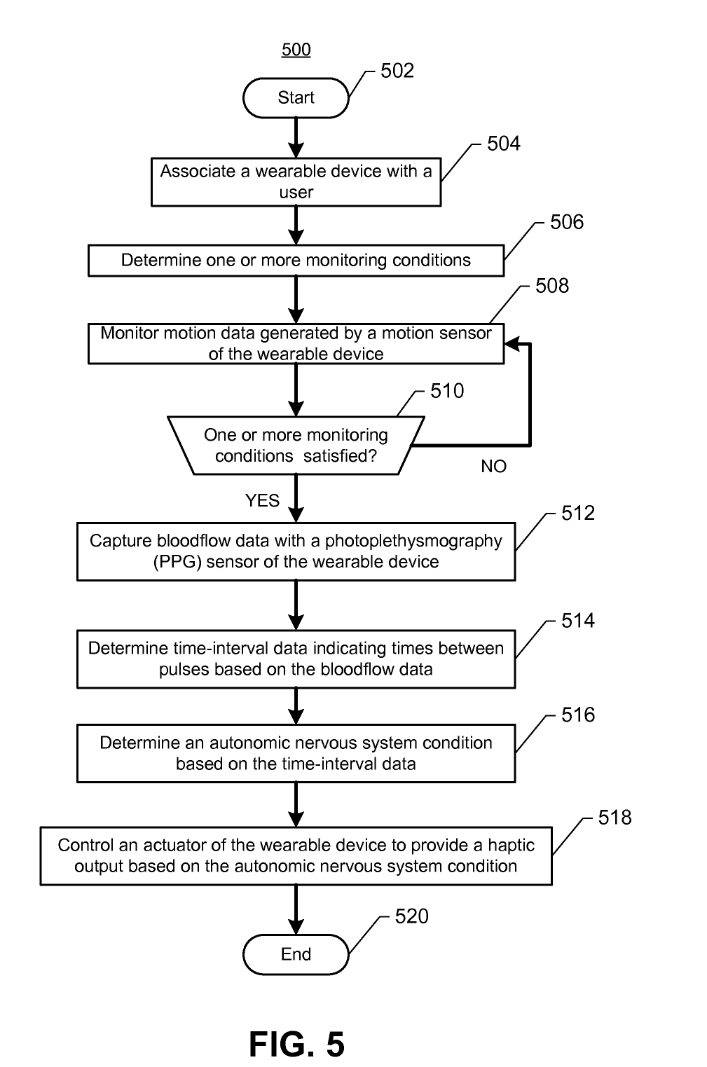

FIG. 5 shows a flow chart of an example of a method 500 for monitoring ANS conditions, in accordance with some embodiments. Method 500 is described as being performed by “processing circuitry,” such as the ANS monitoring circuitry 410 which may be located within the wearable device 110 (e.g., for standalone operation), the user device 108 (e.g., for tethered operation), and/or the server 104 (e.g., for thin client operation of the wearable device 110 as controlled by the server 104 as a remote host).

Method 500 may begin at 502 and proceed to 504, where processing circuitry may be configured to associate a wearable device with a user. The wearable device may include the PPG sensor, motion sensor, display, actuator, etc. as discussed above for the wearable device 300. The processing circuitry may associate the wearable device with the user, such as based on the user creating a user account and/or providing login data (e.g., username, password, biometric identifier, etc.) to the processing circuitry. This may be performed on the user device for a tethered embodiment, and on the wearable device for a standalone embodiment. In the tethered embodiment, the association of the user with the wearable device may be created based on Bluetooth protocol pairing or otherwise connecting the wearable device to the user device after association with the user. Furthermore, the wearable device may be associated with a user account and/or user profile that provides a collection of measured data, programmatically determined data (e.g., ANS conditions) using the measured data, treatments, etc.

At 506, the processing circuitry may be configured to determine one or more monitoring conditions. At 508, the processing circuitry may be configured to monitor motion data generated by the motion sensor of the wearable device. The one or more monitoring conditions may define a physical status of the user that is suitable or optimized for ANS monitoring based on blood flow data. As such, the one or more monitoring conditions may be defined with respect to motion data values or sets/sequences of motion data values over time that are characteristic of targeted user activity, referred to herein as a “motion data fingerprint.” In general, one or multiple monitoring conditions may be used (e.g., concurrently).

In some embodiments, the one or more monitoring conditions may include the user being in a supine or quasi-supine position. This may include the motion data values indicating that the user is oriented in the quasi-supine or supine position as determined by the gyroscope.

In some embodiments, the one or more monitoring conditions may include a lack of motion or substantial motion of the user, indicating that the user is at rest and not partaking in strenuous activity. This may include the motion data values indicating that the user is not in motion, as determined by the accelerator.

In some embodiments, the one or more monitoring conditions may include the user performing spontaneous respiration. Spontaneous respiration refers to natural breathing caused by movement of gas in and out of the lungs that is produced in response to an individual's respiratory muscles. Respiration rate may be measured by a motion sensor disposed at the user's chest wall, which may be included within the wearable device or a separate chest motion sensor that is connected to the wearable device (e.g., worn on the wrist).

At 510, the processing circuitry may be configured to determine whether the one or more monitoring conditions are satisfied based on the monitoring of the motion data. For example, the processing circuitry may control the motion sensor to capture live motion data, and may compare the live motion data to predefined motion data fingerprints. The one or more monitoring conditions may be determined as satisfied if the live motion data matches or corresponds with a predefined motion data fingerprint, and may be determined as unsatisfied if the live motion data fails to match or correspond with the predefined motion data fingerprint(s).

In response to determining that the one or more conditions fail to be satisfied, method 500 may return to 508, where the processing circuitry may be configured to continue monitoring motion data generated by the motion sensor of the wearable device. To provide continuous monitoring for opportunistic capture of blood flow data, the monitoring of motion data and determinations of monitoring condition satisfaction may be performed continuously (e.g., when monitoring is activated) while the wearable device is worn by the user. Advantageously combination of ANS information extraction with motion detection provides for automatically assessing the hemodynamic responses of physiological maneuvers without requiring any participation from the user.

Returning to 510, in response to determining that the one or more monitoring conditions are satisfied, method 500 may proceed to 512, where the processing circuitry may be configured to capture blood flow data with the PPG sensor of the wearable device. The blood flow data refers to a signal generated by the PPG sensor that is indicative of changes in the amount of blood flow over time. In particular, the pulses of blood flow measured by the PPG sensor may be used to determine the user's heartbeat rate because it is the pumping of the blood by the heartbeat which results in the pulses of increased blood flow.

In some embodiments, to reduce power consumption and excessive blood flow data capture, the PPG sensor may be activated only when the one or more monitoring conditions are satisfied. In another example, where the PPG sensor includes multiple LED types (e.g., green and infrared), the higher accuracy but higher power consumption LED (e.g., green) may be activated when the one or more monitoring conditions are satisfied, while the lower accuracy but lower power consumption LED (e.g., red or infrared) may be activated when the one or more monitoring conditions fail to be satisfied.

Advantageously, the wearable device may provide for continuous monitoring of the user throughout daily activity, including rest periods and active periods, and thus does not require the user to specifically set rest time aside for ANS monitoring tasks (e.g., being in a quasi-supine position, and/or not in motion). For example, the processing circuitry may be configured to aggregate blood flow data captured during discontinuous periods of time when the one or more monitoring conditions are satisfied, and use the aggregated blood flow data to perform further processing and ANS condition determinations. Thus, the processing circuitry configured to capture the blood flow data when the one or more monitoring conditions are satisfied may include the processing circuitry being configured to: capture first blood flow data within a first time interval when the one or more monitoring conditions are satisfied, capture second blood flow data within a second time interval when the one or more monitoring conditions are satisfied, wherein the first time interval and the second time interval are discontinuous, and determine the blood flow data based on the first blood flow data and the second blood flow data.

Additionally or alternatively, processing of the blood flow data may performed subsequent the capture of a predetermined amount of blood flow data data points (e.g., 400) sufficient to produce reliable ANS condition determinations. The predetermined amount of blood flow data points may be captured in one continuous period of time, or captured from aggregated blood flow data of multiple discontinuous periods of time. In one embodiment, the wearable device may obtain a first stable measurement through few hundred discontinuous beats worth of data. The device may then update a user profile or user data with every new autonomic information at every “idle”/usable heartbeat (e.g., when the monitoring conditions are satisfied).

At 514, the processing circuitry may be configured to determine time-interval data indicating times between pulses based on the blood flow data. The time-interval data may include a time-varying signal representing a chaotic series of time intervals between quasi-periodical events (e.g., heartbeats represented by the pulses), or pulse-time intervals. The time-interval data may be used to analyze the state of a first system (e.g., the autonomic nervous system) produced by a second system (e.g., the cardiac system) governed by the first system. The quasi-periodical events representing the heartbeat may be detected and the time intervals between these quasi-periodical events may be calculated so as to form the time-interval data or signal. In some embodiments, to generate the time-interval data, signal processing may be applied to the blood flow data, representing a plethysmographic signal. Some example signal processing techniques may include use of a moving-average, adaptive processing using the motion sensor, Kalman, using Principle Component Analysis and/or employing Fourier analysis to evaluate the pulse-time intervals.

At 516, the processing circuitry may be configured to determine an autonomic nervous system condition based on the time-interval data. Such a series of time intervals for the pulses and time-interval data is known to be chaotic. Additional details regarding extracting causal information (e.g., ANS conditions) from chaotic time series data, applicable in some embodiments, is discussed in U.S. Pat. No. 7,092,849, titled “Extracting Causal Information from a Chaotic Time Series,” issued Aug. 15, 2006, which is incorporated by reference herein in its entirety.

For example, determination of the ANS condition may include the processing circuitry being configured to: extract envelope information from the time-interval data or time-varying signal, constructing a phase space for the time-varying signal, extracting information on the relative positions of points corresponding to the time-varying signal in the phase space, combining the envelope and the position information; and based on the combining, providing information relating to the state of the ANS, as discussed in greater detail in U.S. Pat. No. 7,092,849.

Using the techniques discussed in U.S. Pat. No. 7,092,849 and the time-interval data, several parameters may be determined such as the ANSigram₁ defining parasympathetic component values of the ANS, the ANSigram₂ defining sympathetic component values of the ANS, the sympathovagal balance trajectory, the degrees of Autonomic Dysfunction (e.g., wellness) expressed as time proportions, the ANSindex₁ defining parasympathetic index values derived from the ANSigram₁, and ANSindex₂ defining sympathetic index values derived from the ANSigram₂), and the like. As such, the processing circuitry may be configured to: determine sympathetic component values based on the time-interval data, determine sympathetic index values based on the sympathetic component values, determine parasympathetic component values based on the time-interval data, and determine a parasympathetic index values based on the time-interval data. Furthermore, the one or more ANS conditions may be derived from the ANSindex₁ and the ANSindex₂.

Some example ANS conditions may include autonomic dysfunction, wellness, and/or stress. For example, the autonomic dysfunction may represent a lack of coupling between the ANSindex₁ and the ANSindex₂. In general, three distinct behaviors can be observed for the association or coupling between these two indices, as follows:

i) Nearly independent or unrelated behavior: the ANSindex₁ and the ANSindex₂ have values which are significantly different, each evolves in apparent ignorance of the other.

ii) Antagonists: the ANSindex₁ and the ANSindex₂ can reach back near-common values, yet they act in apparent negative-correlation, they also have started to separate scalar-wise.

iii) Interacting: the ANSindex₁ and the ANSindex₂ have mostly common values, they seem to correlate and reflect an interaction between the sympathetic and parasympathetic systems

Autonomic dysfunction measures the lack of association, hence is mainly concerned with behavior that is independent and/or unrelated behavior. The lack of coupling involves a measure of distance between the ANSindex₁ and the ANSindex₂ and indicates a time-proportion for such a distant behavior. The evaluation of autonomic dysfunction may provide an indication of how well homeostasis of the user is maintained.

Wellness measures the desirable interaction or coupling between the sympathetic and parasympathetic systems, and thus the ANSindex₁ and the ANSindex₂. Therefore, it may be concerned with the interacting behavior of these indices. The presence of coupling may include an assessment of correlation, together with the existence of common values. It is appreciated that wellness is not complementary to the autonomic dysfunction because behavior (ii) (e.g., antagonists) often occurs between these indices.

The coupling or lack thereof for the ANSindex₁ and the ANSindex₂ may be determined based on generating a list of distances between the ANSindex₁ and the ANSindex₂ values for a number of time intervals (e.g., a preset time window W, which may include continuous or discontinuous periods of time). A new list value may be generated based on summing the list over the preset time window W to provide a more global measure of the coupling by smoothing out local variations. A ratio between the New List and the window W (New List/w) is then compared to a list threshold value. Weak coupling between the ANSindex₁ and the ANSindex₂ may be determined when the ratio fails to exceed the list threshold value. Conversely, strong coupling between the ANSindex₁ and the ANSindex₂ may be determined when the ratio exceeds the list threshold value.

When evaluating wellness or autonomic dysfunction, a minimal amount of values for ANSindex₁ and the ANSindex₂ may be preferred in order to produce a reliable (i.e., reproducible) estimate. In one example, 400 values may be considered for the ANSindex₁ and the ANSindex₂. Similar to the blood flow data that is used to determine the ANSindex₁ and the ANSindex₂, the ANSindex₁ and the ANSindex₂ may not be required to follow any sort of continuity over a single period of time. For example, discontinuous periods of time within which certain monitoring conditions (e.g., quasi-supine position of the subject and/or spontaneous respiration, etc.) are satisfied may be used. These conditions may not refer to the order or continuity of the extracted ANSindex₁ and the ANSindex₂ values. In some embodiments, discontinuous readings are sufficient for accurately calculating estimates. Moreover, once an estimate is reached, the wellness or autonomic dysfunction values may be updated with each and every new heartbeat captured when the one or more monitoring condition(s) are satisfied.

In some embodiments, a relative position of the user to other users in a population, as the user's wellness or autonomic dysfunction values, can inform about the future health of the user. For example, the distribution of values across a population tends to universally form aggregates (“attractor”) which allow to classify individual users. Moreover, the fact of belonging to a certain class can be: (i) stable, (ii) fast leading to another class in the population (degraded health), and/or (iii) allow reversion to a previous class in the population (improved health). In some embodiments, the aggregation and processing of user data from multiple user may be performed by processing circuitry (e.g., one or more servers) of a central system, such as the central system 102 of the computing system 100/200. In some embodiments, the processing circuitry may be further configured to track, map, and display the user's health time-line (“attractor”).

In some embodiments, the scale of recorded autonomic dysfunction or wellness values may be related with the user's needs for corrective actions (e.g., medical treatments, lifestyle changes, and the like). In some embodiments, using one or more of the above parameters, information corresponding to health and wellness of a user may be derived.

Yet another ANS condition that may be determined includes stress level. The stress level is an example of other health and wellness information that can be extracted from the sympathetic ANSindex₂. For example, an immediate indication of stress (either an absolute threshold or relative to past history (adaptive threshold)) may be received from a user. In some embodiments, the inputs of stress level from the user may be correlated with concurrent ANS parameters, time-interval data, or blood flow data, and subsequent parameters may then be used to programmatically estimate stress.

In general, stress may be considered to be either mental or physical, which may be distinguished based on output of one or more sensors (e.g., the motion sensor) in the wearable device. Stress when the user is stationery or otherwise lacking substantial motion, may be mental (e.g., due to pain/discomfort). On the other hand, when the user is exercising or otherwise in in substantial motion, stress may be considered physical.

In the case of a physical stress, physical activity can be sensed by the motion sensor. In one example, good sympathetic response may be generated due to sports (feedback could be provided). In another example, exaggerated sympathetic response may be avoided (e.g., overtraining syndrome in sprint type sports, when alarms can be provided).

The parasympathetic ANSindex₁ and sympathetic ANSindex₂ can be used individually. In some embodiments, data may be extracted from the parasympathetic ANSindex₁. For example, the following cases may be considered:

Reactivity of the ANSindex₂ to postural changes may be related to emotional regulation i.e., if the parasympathetic ANSindex₁ goes high then the user may be determined as being able to cope with emotions well.

Predominance of the parasympathetic ANSindex₁ may further reveal relaxation. For example, if the parasympathetic ANSindex₁ is high, the subject may be in a very calm, relaxed state.

Patterns in the parasympathetic ANSindex₁ (e.g., plateauing, peaks followed by gradient, etc.) can be identified and be correlated to specific positive emotions (laughter, happiness, etc.) and possibly negative emotions (sadness, weeping, etc.).

Exaggerated parasympathetic response may be identified during workout, as may be determined by the motion data values captured by the motion sensor and/or based on the user activity a workout application or operating mode of the wearable device. For example, this response may be used to prevent overtraining syndrome in endurance sports. In another example, an output (e.g., actuator, speaker, display) can be provided when exaggerated parasympathetic response is detected.

At 518, the processing circuitry may be configured to control the actuator of the wearable device to provide a haptic output based on the autonomic nervous system condition. For example, if autonomic dysfunction or stress values of the user exceeds a predetermined or self-adapting threshold, the device may notify the user with the haptic output generated by the actuator. For example, the processing circuitry may activate a (e.g., linear resonant) actuator 310 the wearable device 300 when the excessive stress, autonomic dysfunction, or a lack of wellness is detected. Advantageously, the wearable device may be attached to the user during daily activity, effectively acting as the user's best buddy and notifying the user discretely to “take it easy” when needed. Other output devices may also be used in addition or alternative to the actuator, such as the display, speaker, etc.

In some embodiments, the wearable device may be configured to facilitate recuperation/recovery from exercise, eating, smoking, heavy drinking, etc. (e.g., traditional recovery). In some embodiments, the wearable device may be additionally or alternatively configured to facilitate recovery through rest, yoga, etc. (e.g., improvement, “negative” recovery). In some embodiments, in order to generate a time of recovery parameter, the processing circuitry may cross-correlate “a posteriori” and “a priori” ANSindex₁ and/or the ANSindex₂ values. In another example, in order to determine a quality of recovery parameter, such as based on relative distance between the ANSindex₁ and the ANSindex₂ values

In some embodiments, a feedback loop may be considered between the indications of wellness, autonomic dysfunction, or stress values and other applications (e.g., Health applications). For example, a user may set goals based on the state of ANS parameters and/or any other estimated parameters, such as time of recovery and the like.

In some embodiments, processing circuitry may monitor fitness, or keep track of trends and/or changes. For example, the wearable device may include a health software application or other application configured to track the health and/or wellness trends of the user. Such tracking and results may be indicated to the user with the various outputs of the wearable device, such as a graphical output on the display, an auditory output from a speaker, or a haptic output from an actuator. Furthermore, monitoring of ANS conditions may be used as feedback for providing guidance for recovery. Method 500 may then proceed to 520 and end.

Many modifications and other example embodiments set forth herein will come to mind to one skilled in the art to which these example embodiments pertain having the benefit of the teachings presented in the foregoing descriptions and the associated drawings. Therefore, it is to be understood that the embodiments are not to be limited to the specific ones disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions other than those explicitly described above are also contemplated as may be set forth in some of the appended claims. Although specific terms are employed herein, they are used in a generic and descriptive sense only and not for purposes of limitation.

That which is claimed:

1. A wearable device, comprising:
a photoplethysmography (PPG) sensor configured to generate blood flow data of user;
a motion sensor configured to generate motion data indicating motion of the user; and
processing circuitry configured to:
determine one or more monitoring conditions;
determine whether the one or more monitoring conditions are satisfied based on monitoring the motion data;
capture the blood flow data when the one or more monitoring conditions are satisfied;
determine time-interval data indicating times between pulses based on the blood flow data; and
determine an autonomic nervous system condition based on the time-interval data.

2. The wearable device of claim 1, wherein:
the processing circuitry configured to capture the blood flow data when the one or more monitoring conditions are satisfied includes the processing circuitry being configured to:
capture first blood flow data within a first time interval when the one or more monitoring conditions are satisfied;
capture second blood flow data within a second time interval when the one or more monitoring conditions are satisfied, wherein the first time interval and the second time interval are discontinuous; and
determine the blood flow data based on aggregating the first blood flow data and the second blood flow data.

3. The wearable device of claim 1, wherein:
the processing circuitry configured to capture the blood flow data when the one or more monitoring conditions are satisfied includes the processing circuitry being configured to capture blood flow data within discontinuous time intervals until a predetermined amount of the blood flow data sufficient for the autonomous nervous condition determination has been captured while the one or more monitoring conditions are satisfied.

4. The wearable device of claim 1, wherein the one or more monitoring conditions include at least one of:
the user being in a quasi-supine position;
the user performing spontaneous respiration; or
a lack of user motion.

5. The wearable device of claim 1, wherein the processing circuitry is further configured to:
determine sympathetic component values based on the time-interval data;
determine sympathetic index values based on the sympathetic component values;
determine parasympathetic component values based on the time-interval data; and
determine a parasympathetic index values based on the time-interval data.

6. The wearable device of claim 5, wherein the processing circuitry configured to determine the autonomic nervous system condition includes the processing circuitry being configured to determine an autonomic dysfunction indicating a lack of coupling between the sympathetic index values and the parasympathetic index values.

7. The wearable device of claim 5, wherein the processing circuitry configured to determine the autonomic nervous system condition includes the processing circuitry being configured to determine a wellness value indicating a desirable coupling between the sympathetic index values and the parasympathetic index values.

8. The wearable device of claim 5, wherein:
the one or more monitoring conditions include the user being stationary;
the wearable device further includes an actuator configured to provide a haptic output; and
the processing circuitry configured to determine the autonomic nervous system condition includes the processing circuitry being configured to:
determine a stress value based on the sympathetic index values;
determine a stress value threshold; and
control the actuator to provide the haptic output when the stress value satisfies the stress value threshold.

9. The wearable device of claim 1, wherein the processing circuitry is further configured to determine a time of recovery parameter or a quality of recovery parameter based on at least one of the ANSindex₁ or the ANSindex₂.

10. The wearable device of claim 1, wherein:
the wearable device further includes an actuator configured to provide a haptic output; and
the processing circuitry is further configured to control the actuator to provide the haptic output based on the autonomic nervous system condition.

11. The wearable device of claim 1, wherein the photoplethysmography (PPG) sensor includes:
one or more light emitting diodes (LEDs) configured to provide light on the user's skin; and
one or more photodiodes configured to measure changes in absorption or reflection of the light by the user's skin to generate the blood flow data of the user.

12. The wearable device of claim 11, wherein the one or more LEDs include at least one of:
a green LED;
a red LED;
a near-infrared LED;
an infrared LED; or
a blue LED.

13. The wearable device of claim 11, wherein:
the one or more LEDs include a green LED and an infrared LED; and
the processing circuitry is further configured to:
capture the blood flow data when the one or more monitoring conditions are satisfied using the green LED to provide the light on the user's skin; and
capture second blood flow data when the one or more monitoring conditions fails to be satisfied using the infrared LED to provide the light on the user's skin.

14. A method for autonomic nervous system (ANS) monitoring, comprising:
determining, by processing circuitry of an apparatus, one or more monitoring conditions;
determining, by the processing circuitry, whether the one or more monitoring conditions are satisfied based on monitoring motion data captured by a motion sensor of a wearable device;
controlling, by the processing circuitry, a photoplethysmography (PPG) sensor of the wearable device to capture blood flow data when the one or more monitoring conditions are satisfied;
determining, by the processing circuitry, time-interval data indicating times between pulses based on the blood flow data; and
determining, by the processing circuitry, an autonomic nervous system condition based on the time-interval data.

15. The method of claim 14, wherein controlling the PPG sensor to capture the blood flow data when the one or more monitoring conditions are satisfied includes:
controlling the PPG sensor to capture first blood flow data within a first time interval when the one or more monitoring conditions are satisfied;
controlling the PPG sensor to capture second blood flow data within a second time interval when the one or more monitoring conditions are satisfied, wherein the first time interval and the second time interval are discontinuous; and
determining the blood flow data based on aggregating the first blood flow data and the second blood flow data.

16. The method of claim 14, wherein:
controlling the PPG sensor to capture the blood flow data when the one or more monitoring conditions are satisfied includes controlling the PPG sensor to capture blood flow data within discontinuous time intervals until a predetermined amount of the blood flow data sufficient for the autonomous nervous condition determination has been captured while the one or more monitoring conditions are satisfied.

17. The method of claim 14, wherein the one or more monitoring conditions include at least one of:
the user being in a quasi-supine position;
the user performing spontaneous respiration; or
a lack of user motion.

18. The method of claim 14 further comprising, by the processing circuitry:
determining sympathetic component values based on the time-interval data;
determining sympathetic index values based on the sympathetic component values;
determining parasympathetic component values based on the time-interval data; and
determining a parasympathetic index values based on the time-interval data.

19. The method of claim 18, wherein determining the autonomic nervous system condition includes determining an autonomic dysfunction indicating a lack of coupling between the sympathetic index values and the parasympathetic index values.

20. The method of claim 18, wherein determining the autonomic nervous system condition includes determining a wellness value indicating a desirable coupling between the sympathetic index values and the parasympathetic index values.

21. The method of claim 14, wherein:
the one or more monitoring conditions include the user being stationary;
the wearable device further includes an actuator configured to provide a haptic output; and
determining the autonomic nervous system condition includes:
determining a stress value based on the sympathetic index values;
determining a stress value threshold; and
controlling the actuator to provide the haptic output when the stress value satisfies the stress value threshold.

22. The method of claim 14 further comprising, by the processing circuitry, determining a time of recovery parameter or a quality of recovery parameter based on at least one of the ANSindex₁ or the ANSindex₂.

22. The method of claim 14, wherein:
the wearable device further includes an actuator configured to provide a haptic output; and
the method further includes, by the processing circuitry, controlling the actuator to provide the haptic output based on the autonomic nervous system condition.

23. The method of claim 14, wherein the photoplethysmography (PPG) sensor includes:
one or more light emitting diodes (LEDs) configured to provide light on the user's skin; and
one or more photodiodes configured to measure changes in absorption or reflection of the light by the user's skin to generate the blood flow data of the user.

24. The method of claim 23, wherein the one or more LEDs include at least one of:
a green LED;
a red LED;
a near-infrared LED;
an infrared LED; or
a blue LED.

25. The method of claim 23, wherein:
the one or more LEDs include a green LED and an infrared LED; and
the processing circuitry is further configured to:
capture the blood flow data when the one or more monitoring conditions are satisfied using the green LED to provide the light on the user's skin; and
capture second blood flow data when the one or more monitoring conditions fails to be satisfied using the infrared LED to provide the light on the user's skin.

26. The method of claim 14, wherein the apparatus including the processing circuitry is one of:
the wearable device;
a user device tethered to the wearable device; or
a server connected to the wearable device via the Internet.
